(19) United States
(12) Patent Application Publication
(10) Pub. No.: US 2017/0086791 A1
(43) Pub. Date: Mar. 30, 2017

(54) APPARATUS AND METHOD FOR SUPPORTING ACQUISITION OF AREA-OF-INTEREST IN ULTRASOUND IMAGE

(71) Applicant: SAMSUNG ELECTRONICS CO., LTD., Suwon-si (KR)

(72) Inventors: Seung-Chul CHAE, Seoul (KR); Yeong-Kyeong SEONG, Yongin-si (KR)

(73) Assignee: SAMSUNG ELECTRONICS CO., LTD., Suwon-si (KR)

(21) Appl. No.: 15/310,975

(22) PCT Filed: Jun. 25, 2014
(86) PCT No.: PCT/KR2014/005643

§ 371 Date: Nov. 14, 2016

(51) Int. Cl. A61B 8/00; A61B 6/00; G06K 9/46; G06T 7/00; G06T 7/11; A61B 8/08; A61B 8/13

(52) U.S. Cl. CPC A61B 8/469; A61B 8/5261; A61B 8/466; A61B 6/502; A61B 8/0825; A61B 8/13; G06T 7/0012; G06T 7/11; G06K 9/4604; A61B 2090/367

(57) ABSTRACT

The present disclosure relates to an apparatus for supporting acquisition of an area-of-interest in an ultrasound image. An apparatus for supporting acquisition of an area-of-interest in an ultrasound image according to an aspect of the present disclosure may comprise: at least one processor configured to convert coordinates of one or more areas-of-interest extracted from a first image to coordinates on a 3D model and collect the coordinates on the 3D model of the one or more areas-of-interest; when a second image is acquired, calculate coordinates on the 3D model corresponding to the acquired second image and match the second image onto the 3D model including the coordinates of the collected areas-of-interest; and provide guide information to allow acquisition of the collected areas-of-interest from the second image, using the matching result.

### TECHNICAL FIELD

The present disclosure relates to technology pertaining to an apparatus and a method for supporting acquisition of an area-of-interest in an ultrasound image.

### BACKGROUND ART

Typically, a primary screening is performed by radiography, and a secondary diagnosis and a definite diagnosis are made by ultrasonography.

Mammography refers to positioning a breast between two units of examination equipment and then taking X-ray photographs of the breast while pressure is applied on the breast, and typically takes two types of photographs from top to bottom and from left to right and a specialist reads the photographs. However, the mammography is less efficient for young women or women with dense breast tissue, and thus it is difficult to detect all the cancers in time only with X-rays. Also, although a tumor or a calcification lesion is visible in an image obtained by the mammography, it is difficult to accurately know the relevant area. Therefore, for surgery, it is necessary to again find the location of the tumor through ultrasound.

Breast ultrasonography is a diagnosis method capable of easily and rapidly acquiring images from various angles and immediately checking the acquired images. Also, the breast ultrasonography uses sound waves of high frequency, and thus, is harmless to humans and more easily finds a lesion than the mammography with respect to a highly dense breast.

Typically, a primary screening is performed by the mammography, and a secondary diagnosis and a definite diagnosis are made by the breast ultrasonography. However, differently from X-ray images which show even results over all the images, in the ultrasonography, an image shown according to a time point and an angle becomes distinctly different depending on a probe manipulated by an ultrasonographer. Therefore, the ultrasonography is problematic in that acquired images are different depending on subjectivity and the degree of proficiency of the ultrasonographer. Also, typically, the breast ultrasonography which is a secondary examination is performed by clinical judgment of a specialist on the basis of the prior information acquired by the mammography which is a primary examination. Therefore, a conventional method is problematic in that it is difficult to obtain objective and accurate results.

### DETAILED DESCRIPTION OF THE INVENTION

### Technical Problem

Proposed are an apparatus and a method for providing a guide so as to acquire a more precise area-of-interest in an ultrasound image with respect to an area-of-interest acquired in an X-ray image.

### Technical Solution

In accordance with an aspect of the present disclosure, an apparatus for supporting acquisition of an area-of-interest in an ultrasound image is provided. The apparatus may include at least one processor configured to convert coordinates of one or more areas-of-interest extracted from a first image into coordinates on a three-dimensional (3D) model, and collect coordinates on the 3D model of the one or more areas-of-interest; calculate coordinates on the 3D model corresponding to an acquired second image when the second image is acquired, and match the second image onto the 3D model including the collected coordinates of the areas-of-interest; and provide guide information so as to acquire the collected areas-of-interest in the second image, by using a result of the matching.

At this time, the first image may correspond to a radiographic image, and the second image may correspond to an ultrasonographic image.

When the first image corresponds to a 3D image, the at least one processor may convert a coordinate system of a measurement device, that has captured the first image, into a coordinate system of the 3D model, and thereby may convert the coordinates of the one or more areas-of-interest into the coordinates on the 3D model.

When the first image corresponds to two or more 2D cross-sectional images obtained by image-capturing an identical area-of-interest, the at least one processor may collect, as the coordinates of the area-of-interest, coordinates of an intersection point of straight lines on the 3D model which are respectively formed perpendicular to the cross-sectional images corresponding to the 2D coordinates of the area-of-interest, which is included in at least some of the two or more 2D cross-sectional images, with the 2D coordinates of the area-of-interest as a center.

When the first image corresponds to a 2D image and an intersection point is not formed based on coordinates of an area-of-interest included in at least some 2D cross-sectional images among the one or more first images, the at least one processor may determine, as an area-of-interest, a predetermined area which is converted into coordinates on the 3D model with the coordinates of the area-of-interest, which is included in the at least some 2D cross-sectional images, as a center.

At this time, the predetermined area may include one or more of: straight lines which are converted into coordinates on the 3D model and which are respectively formed perpendicular to cross-sectional images corresponding to the coordinates of the area-of-interest, which is included in the at least some 2D cross-sectional images, with the coordinates of the area-of-interest as the center; and an area within a preset radius with the converted coordinates of the areas-of-interest as a center.

Also, the apparatus may further include a display configured to output the 3D model on a screen, and to display the one or more areas-of-interest on the output 3D model in a preset form including one or more of a point, a line, a plane, and a polygon based on the collected coordinates of the areas-of-interest.

The display may display the 3D model in at least one form among preset forms including one or more of translucency and a contour.

The at least one processor may collect area-of-interest information including one or more of the number of the one or more areas-of-interest, grades thereof, types thereof, and attributes thereof, and the display may further display the collected area-of-interest information at a predetermined position on the 3D model.

The at least one processor may determine whether an area-of-interest exists which has not been acquired in the second image among the one or more areas-of-interest, based on the converted coordinates of the areas-of-interest and the calculated coordinates of the second image, and may provide the guide information to a user so as to acquire a new second image, when it is determined that the area-of-interest exists which has not been acquired in the second image.

The at least one processor may display an arrow indicating an area-of-interest which has not been acquired in the second image, or may emphasize and display the area-of-interest, which has not been acquired in the second image, by using one or more of a type of an edge color of an outline, a line type of the outline, and a line thickness of the outline.

The guide information may include one or more pieces of information among an order of the acquisition of the areas-of-interest, position information of the second image, a degree of proximity of the second image to the areas-of-interest, a progress direction of the second image, the number of areas-of-interest which have not been acquired, and position information of the areas-of-interest which have not been acquired.

In accordance with an aspect of the present disclosure, a method for supporting acquisition of an area-of-interest in an ultrasound image is provided. The method may include converting coordinates of one or more areas-of-interest extracted from a first image into coordinates on a three-dimensional (3D) model, and collecting coordinates on the 3D model of the one or more areas-of-interest; calculating coordinates on the 3D model corresponding to an acquired second image when the second image is acquired; matching the second image onto the 3D model including the collected coordinates of the areas-of-interest; and providing guide information to a user so as to acquire the one or more areas-of-interest in the second image, by using a result of the matching.

The collecting of the coordinates on the 3D model of the one or more areas-of-interest may include, when the first image corresponds to a 3D image, converting a coordinate system of a measurement device, that has captured the first image, into a coordinate system of the 3D model, and thereby converting the coordinates of the one or more areas-of-interest into the coordinates on the 3D model.

The collecting of the coordinates on the 3D model of the one or more areas-of-interest may include, when the first image corresponds to two or more 2D cross-sectional images obtained by image-capturing an identical area-of-interest, collecting, as the coordinates of the area-of-interest, coordinates of an intersection point of straight lines on the 3D model which are respectively formed perpendicular to cross-sectional images corresponding to the 2D coordinates of the area-of-interest, which is included in at least some of the two or more 2D cross-sectional images, with the 2D coordinates of the area-of-interest as a center.

The collecting of the coordinates on the 3D model of the one or more areas-of-interest may include, when the first image corresponds to a 2D image and an intersection point is not formed based on coordinates of an area-of-interest included in at least some 2D cross-sectional images among the one or more first images, determining, as an area-of-interest, a predetermined area which is converted into coordinates on the 3D model with the coordinates of the area-of-interest, which is included in the at least some 2D cross-sectional images, as a center.

At this time, the predetermined area may include one or more of: straight lines which are converted into coordinates on the 3D model and which are respectively formed perpendicular to cross-sectional images corresponding to the coordinates of the area-of-interest, which is included in the at least some 2D cross-sectional images, with the coordinates of the area-of-interest as the center; and an area within a preset radius with the converted coordinates of the areas-of-interest as a center.

Also, the method may further include outputting the 3D model on a screen; and displaying the one or more areas-of-interest on the output 3D model in a preset form including one or more of a point, a line, a plane, and a polygon based on the collected coordinates of the areas-of-interest.

The collecting of the coordinates on the 3D model of the one or more areas-of-interest may include collecting area-of-interest information including one or more of the number of the one or more areas-of-interest, grades thereof, types thereof, and attributes thereof; and the displaying of the one or more areas-of-interest may include further displaying the collected area-of-interest information at a predetermined position on the 3D model.

The providing of the guide information may include: determining whether an area-of-interest exists which has not been acquired in the second image among the one or more areas-of-interest, based on the converted coordinates of the areas-of-interest and the calculated coordinates of the second image; and providing a guide so as to acquire a new second image, when it is determined that the area-of-interest exists which has not been acquired in the second image.

The providing of the guide information may include displaying an arrow indicating an area-of-interest which has not been acquired in the second image, or emphasizing and displaying one or more of a type of an edge color of an outline of the area-of-interest which has not been acquired in the second image, a line type of the outline of the area-of-interest, and a line thickness of the outline of the area-of-interest.

### Advantageous Effects

Support can be provided to acquire a more precise area-of-interest in an ultrasound image with respect to an area-of-interest acquired in an X-ray image, and thereby, an omission can be prevented in acquiring an area-of-interest and an effect exerted by an ultrasonographer can be reduced. Therefore, objective and accurage results can be obtained.

### BRIEF DESCRIPTION OF THE DRAWINGS

FIG. 1 is a block diagram illustrating an apparatus for supporting acquisition of an area-of-interest in an ultrasound image according to an embodiment of the present disclosure.

FIG. 2 illustrates an example of reading an area-of-interest in an X-ray image.

FIG. 3A illustrates an example of converting coordinates of an area-of-interest into a three-dimensional (3D) model.

FIG. 3B illustrates an example of matching a second image onto a 3D model including coordinates of an area-of-interest.

FIG. 4 illustrates an example of displaying position information of an ultrasound image on an ultrasonographic image and a 3D model.

FIG. 5 is a flowchart illustrating a method for supporting acquisition of an area-of-interest in an image according to an embodiment of the present disclosure.

FIG. 6A illustrates an example of displaying an area-of-interest on a translucent 3D model.

FIG. 6B illustrates an example of providing guide information on a progress direction of a second image onto a 3D model having an area-of-interest displayed thereon.

### MODE FOR CARRYING OUT THE INVENTION

Other details of embodiments are included in the detailed description and the drawings. The advantages and features of the present disclosure and methods of achieving the same will be apparent by referring to embodiments of the present disclosure as described below in detail in conjunction with the accompanying drawings. Throughout the specification, the same or like reference numerals designate the same or like elements.

Hereinafter, embodiments of an apparatus and a method for supporting analysis of a 3D ultrasound image will be described in detail with reference to the accompanying drawings.

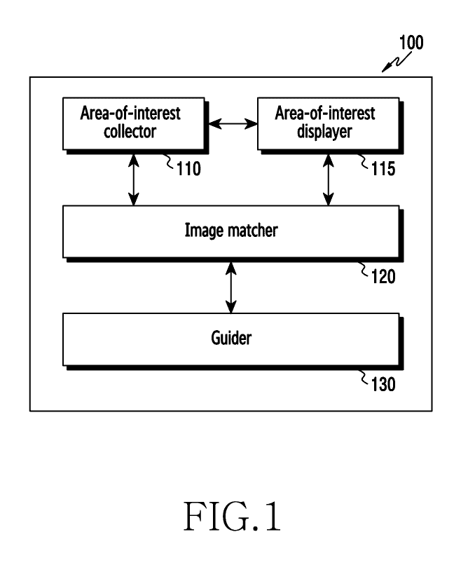

FIG. 1 is a block diagram illustrating an apparatus 100 for supporting acquisition of an area-of-interest in an ultrasound image according to an embodiment of the present disclosure. Referring to FIG. 1, the apparatus 100 for supporting acquisition of an area-of-interest in an ultrasound image may include an area-of-interest collector 110, an area-of-interest displayer 115, an image matcher 120, and a guider 130.

According to an embodiment of the present disclosure, an X-ray image is a first image and an ultrasonographic image is a second image, and support may be provided to acquire an area-of-interest in the second image. An area which is seen as a lesion area in the X-ray image may be designated as an area-of-interest, and the area-of-interest is read from the X-ray image. At this time, the X-ray image may have two-dimensional (2D) or 3D coordinates.

The area-of-interest collector 110 may convert coordinates of the area-of-interest, which is extracted from the first image, into coordinates on a 3D model. One or more areas-of-interest may exist, and the area-of-interest collector 110 may collect the coordinates on the 3D model of the extracted area-of-interest.

When the first image is a 3D image, the area-of-interest collector 110 may convert the coordinates of the area-of-interest into coordinates on the 3D model by converting a coordinate system of a measurement device, that has captured the first image, into a coordinate system of the 3D model.

When the first image is a 2D image, the area-of-interest collector 110 may convert 2D coordinates of an area-of-interest, which is included in a 2D cross-sectional image, into coordinates on a 3D model. When there are two or more cross-sectional images obtained by image-capturing an identical area-of-interest, the area-of-interest collector 110 may collect, as coordinates of the area-of-interest, coordinates of an intersection point of straight lines on the 3D model which are respectively formed perpendicular to the cross-sectional images corresponding to the 2D coordinates of the area-of-interest with the 2D coordinates of the area-of-interest as a center.

The first image has a 2D coordinate system, and thus lacks information of one axis among x, y, and z of a 3D coordinate system. Accordingly, when (x1, y1) representing the coordinates of the area-of-interest extracted from the 2D first image are converted into coordinates of the 3D coordinate system, (x1, y1, z) is obtained. (x1, y1, z) may form a straight line which is vertical in the z-axis direction on the x-y plane. Accordingly, the coordinates of the area-of-interest extracted from the two or more first images obtained by image-capturing the identical area-of-interest may be converted into coordinates of the 3D coordinate system, an intersection point of straight lines which are respectively perpendicular to the cross-sectional images may be found, and thereby, coordinates of the area-of-interest on the 3D model may be calculated.

In contrast, if an intersection point is not formed when the coordinates of the identical area-of-interest extracted from the two or more 2D cross-sectional images are converted into 3D coordinates, the area-of-interest collector 110 may not collect exact coordinates of the area-of-interest on the 3D model. In this case, coordinates of areas-of-interest included in some of the cross-sectional images are respectively converted into coordinates on the 3D model, and a predetermined area is determined as an area-of-interest, with the converted coordinates on the 3D model of the areas-of-interest as a center. When conversion from 2D coordinates to 3D coordinates is performed based on a pair of 2D coordinates extracted from the first image, a predetermined area may also be determined as an area-of-interest, with coordinates on the 3D model of the area-of-interest as a center.

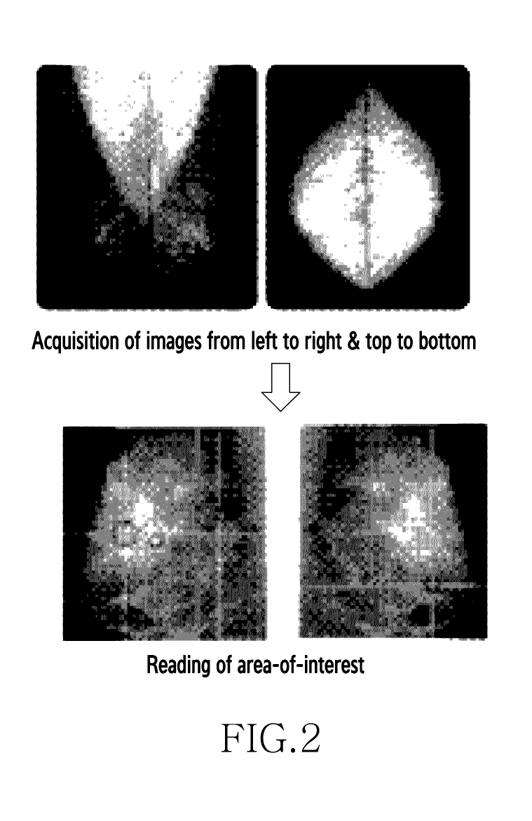

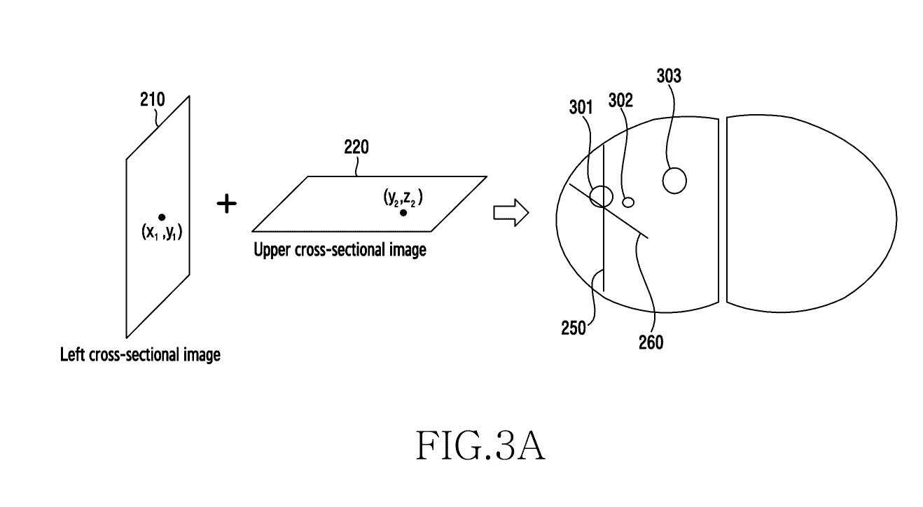

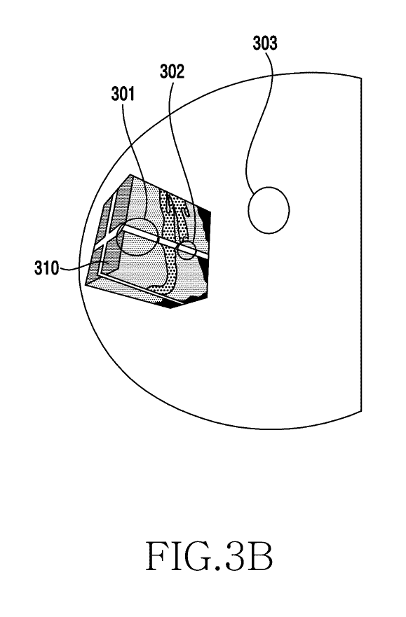

At this time, the predetermined area may be a straight line formed perpendicular to one 2D cross-sectional image, with coordinates of an area-of-interest included in the 2D cross-sectional image as a center, and may be an area within a preset radius, with the converted coordinates of the area-of-interest on the 3D model as a center. An example of a preset form may variously exist. An embodiment of collecting an area-of-interest in a case where an X-ray image is the first image will be described with reference to FIGS. 2 and 3.

Meanwhile, the area-of-interest collector 110 may receive, as input, information on an area-of-interest from a user; and may receive coordinates of an area-of-interest extracted from the first image, may read an area-of-interest from the first image and may extract the area-of-interest, and may calculate coordinates of the area-of-interest in the first image. However, the present disclosure is not limited thereto.

Also, the area-of-interest collector 110 may collect area-of-interest information, such as the number of areas-of-interest, a grade of an area-of-interest, a type thereof, an attribute thereof, and the like. According to an embodiment of the present disclosure, the area-of-interest collector 110 may further collect additional information on an area-of-interest, such as a grade which is assigned according to the classification of areas-of-interest in order of importance, a type depending on a form of an area-of-interest, an attribute of an area-of-interest depending on prior information which is speculated from the size, color, form, and the like of a lesion area, and the like.

The area-of-interest displayer 115 may output a 3D model on a screen, and may display the 3D model in a translucent form or in the form of a contour. Also, the area-of-interest displayer 115 may display the 3D model so as to be proportional to a human body. The area-of-interest displayer 115 may display an area-of-interest by using a point, a line, a plane, a polygon, and the like on the basis of coordinates on the 3D model of the area-of-interest. At this time, an area-of-interest may be determined in the form of a cylinder having a radius R, with the coordinates of the area-of-interest converted into the 3D model as a center. Here, only the forms of a point and a line are described, but the areas-of-interest may be displayed in forms of a sphere having a predetermined radius and a polygon.

Also, the area-of-interest may be emphasized and displayed on the 3D model in such a manner as to highlight and display the area-of-interest, to attach a marker to the area-of-interest, or the like. In addition, the collected area-of-interest information may be further displayed at a predetermined position on the 3D model.

For example, coordinates of the area-of-interest may be displayed on the 3D model, the area-of-interest may be emphasized and displayed by attaching a marker to the displayed coordinates, and information on a grade, a type, an attribute, and the like of the area-of-interest may be displayed together. The area-of-interest information may be output on the 3D model, or may be output as additional information on a screen which is distinguished from the 3D model.

Typically, an ultrasound measurement device performs a diagnosis by using a probe, and acquires, in real time, an image obtained by image-capturing an affected area of a patient. Accordingly, the position and direction of the probe need to be modified in real time in order to acquire a high-quality image for accurate treatment.

The second image may be acquired from the ultrasound measurement device. When the second image is acquired, the image matcher 120 calculates coordinates on the 3D model corresponding to the acquired second image. Then, the image matcher 120 may match the second image onto the 3D model including coordinates on the 3D model of an area-of-interest. At this time, the image matcher 120 may match the acquired second image onto the 3D model, or may match a predetermined area, which is obtained by calculating the coordinates on the 3D model corresponding to the acquired second image, onto the 3D model. Since the position of the second image may be changed in real time, the matched coordinates on the 3D model of the second image may be newly calculated according to the position change of the second image, and the changed position of the second image may be reflected on the 3D model.

At this time, the coordinates on the 3D model corresponding to the second image may be received as input from the user, or may be received from a device that collects the second image.

The guider 130 may provide guide information so as to acquire the second image including the area-of-interest, by using a result of the matching. At this time, a method for displaying the second image and the area-of-interest may be diversified to be capable of identifying the second image and the area-of-interest. For example, by using the result of the matching, the guider 130 may dispay the acquired second image and the collected areas-of-interest in such a manner as to overlay the acquired second image with the collected areas-of-interest on the 3D model. This configuration may help to determine whether the areas-of-interest have been acquired in the second image.

The guider 130 may determine an area-of-interest, which has not been acquired in the second image among the collected areas-of-interest, on the basis of the converted coordinates of the area-of-interest and the calculated coordinates of second image. When it is determined that the area-of-interest exists which has not been acquired in the second, the guider 130 may provide guide information to the user so as to acquire a new second image.

Various embodiments of providing the guide information to the user may be implemented. For example, an area-of-interest may be marked and displayed on the 3D model, and a determination may be made as to whether an area-of-interest has been acquired in an acquired second image. At this time, since the position of the second image may be changed, a guide is provided so that the user may change the position of the second image and may acquire an area-of-interest.

As an example, the guider 130 may display an arrow indicating the area-of-interest which has not been acquired in the second image, or may emphasize and display one or more of the type of edge color of an outline of the area-of-interest, a line type of the outline of the area-of-interest, and a line thickness of the outline of the area-of-interest.

According to an embodiment of the present disclosure, when the number of areas-of-interest is plural, an order of areas-of-interest required to be acquired may be calculated according to a grade of an area-of-interest, an importance thereof, an attribute thereof, a type thereof, and the like, and the calculated order may be provided to the user.

Also, the guide information provided to the user may include an order of acquisition of areas-of-interest, position information of the second image, the degree of proximity of the second image to an area-of-interest, a progress direction of the second image, the number of areas-of-interest which have not been acquired, and position information of the areas-of-interest which have not been acquired.

The area-of-interest may be marked and displayed, or may include additional information therein and may be provided to the user in a state of including the additional information; and may have the degree of proximity to an area-of-interest according to the proximity of the second image to the area-of-interest, a current progress direction of the second image, and a guided progress direction of the second image which are displayed on the area-of-interest. Also, a determination may be made as to whether an area-of-interest has been acquired in the second image; the user may be provided with the number of areas-of-interest which have not been acquired, coordinate information on an area-of-interest required to be acquired next, and the like; and position information of the areas-of-interest which have not been acquired may be guided to the user.

FIG. 2 illustrates an example of reading an area-of-interest in an X-ray image.

Referring to FIG. 2, a first image may be a 2D cross-sectional image obtained by image-capturing a breast by using X-rays used in a breast examination, and may be cross-sectional images obtained by image-capturing the breast from left, right, top, and bottom directions. When an area-of-interest suspected as a lesion area exists in each cross-sectional image, the area-of-interest is extracted. Referring to FIG. 2, the area-of-interest is read from the X-ray image, and coordinates of the area-of-interest may be extracted. A part indicated by a circle in the lower view may be designated as an area-of-interest and coordinates of the area-of-interest may be extracted with reference to a vertical straight line and a horizontal straight line in the area-of-interest. In this example, the X-ray image has 2D coordinates, and thus, the extracted area-of-interest may also have 2D coordinates. One or more areas-of-interest may exist, and in this example, three areas-of-interest may be extracted from the left cross-sectional image of the breast, and one area-of-interest may be extracted from the right cross-sectional image of the breast.

Hereinafter, an example in which the area-of-interest collector 110 illustrated in FIG. 1 converts coordinates of an area-of-interest extracted from an X-ray image into coordinates on a 3D model and collects coordinates on the 3D model corresponding to the area-of-interest will be described with reference to FIG. 3A.

FIG. 3A illustrates an example of converting coordinates of an area-of-interest into a three-dimensional (3D) model.

As exemplified in FIG. 3A, when radiographic images are collected which are obtained by image-capturing the human body and have 2D coordinates from top to bottom and from left to right, if an area-of-interest extracted from a left cross-sectional image 210 from among the radiographic images is converted into coordinates on a 3D model, the area-of-interest may be formed on the 3D model in the form of a straight line 260 perpendicular to the left cross-sectional image. The area-of-interest exists on the straight line 260, but since a pair of exact coordinates may not be recognized on the 3D model, an area-of-interest needs to be acquired by inspecting the entire straight line. Accordingly, an area of the straight line on the 3D model may be determined as a new area-of-interest.

When an identical area-of-interest exists in the left cross-sectional image 210 and an upper cross-sectional image 220, an intersection point may be obtained by converting the identical area-of-interest into a 3D model, and the intersection point may be collected as coordinates of the area-of-interest on the 3D model. When the left cross-sectional image 210 has an x-y plane and the upper cross-sectional image 220 has an y-z plane, (x1, y1) representing 2D coordinates of an area-of-interest extracted from the left cross-sectional image 210 and (y2, z2) representing 2D coordinates of an area-of-interest extracted from the upper cross-sectional image 220 may be calculated. When (x1, y1) and (y2, z2) are converted into coordinates on the 3D model, the coordinates of the area-of-interest extracted from the left cross-sectional image may be converted into the straight line 260 having coordinates of (x1, y1, z). Also, the coordinates of the area-of-interest extracted from the upper cross-sectional image 220 may be converted into the straight line 250 having coordinates of (x, y2, z2). Referring to FIG. 3, an intersection point formed by the two straight lines 250 and 260 may be determined as coordinates of an area-of-interest 301 on the 3D model. Areas-of-interest 301, 302, and 303 may be displayed in the form of including a predetermined area on the 3D model.

However, there may be a case where the amount of information is insufficient, such as a case, in which an area-of-interest is read from the left cross-sectional image but an area-of-interest is not read from the upper cross-sectional image, and the like. In this case, since an intersection point is not formed based on 2D coordinates of an area-of-interest, coordinates of an area-of-interest included in at least some of the 2D cross-sectional images are converted into coordinates on the 3D model, and a predetermined area may be determined as an area-of-interest, with the converted coordinates on the 3D model as a center. The predetermined area may be an area having a radius R with the converted coordinates as a center. Alternatively, the predetermined area may have a form, such as a point, a line, a plane, a polygon, a sphere, and the like which have a predetermined area according to data preset by the user.

FIG. 3B illustrates an example of matching a second image onto a 3D model including coordinates of an area-of-interest. A shaded part is the 3D model. The apparatus 100 for supporting acquisition of an area-of-interest in an ultrasound image may collect coordinates of areas-of-interest on the 3D model and may output the collected coordinates of the areas-of-interest. In FIG. 3B, three areas-of-interest 301, 302, and 303 are each represented in the form of a circle. Also, the apparatus 100 may calculate coordinates on the 3D model of the acquired second image, and may match position information of the second image on the 3D model to the 3D model. At this time, the position information of the second image on the 3D model may be matched onto the 3D model in a predetermined form representing an image-capturing range of the second image, and referring to FIG. 3B, may be matched onto the 3D model having a 3D rectangular shape 310.

The guider 130 illustrated in FIG. 1 provides guide information to the user on the basis of a result of the matching. In an example of FIG. 3B, the acquired second image and the collected areas-of-interest are represented in such a manner as to overlay the acquired second image with the collected areas-of-interest on the 3D model. Also, it can be confirmed that in the second image 310, the two areas-of-interest 301 and 302 have been acquired and one area-of-interest 303 has not been acquired. The user may be guided by the apparatus 100 for supporting acquisition of an area-of-interest in an ultrasound image so as to acquire the one area-of-interest 303, which has not been acquired, in the second image 310, and may change the position of the second image. For example, the apparatus 100 for supporting acquisition of an area-of-interest in an ultrasound image may provide the user with coordinate information in a direction in which the second image needs to progress, or may display an area-of-interest in such a manner as to mark and emphasize the area-of-interest. The changed position of the second image may be recalculated by the apparatus 100 for supporting acquisition of an area-of-interest in an ultrasound image, and may be reflected on the 3D model.

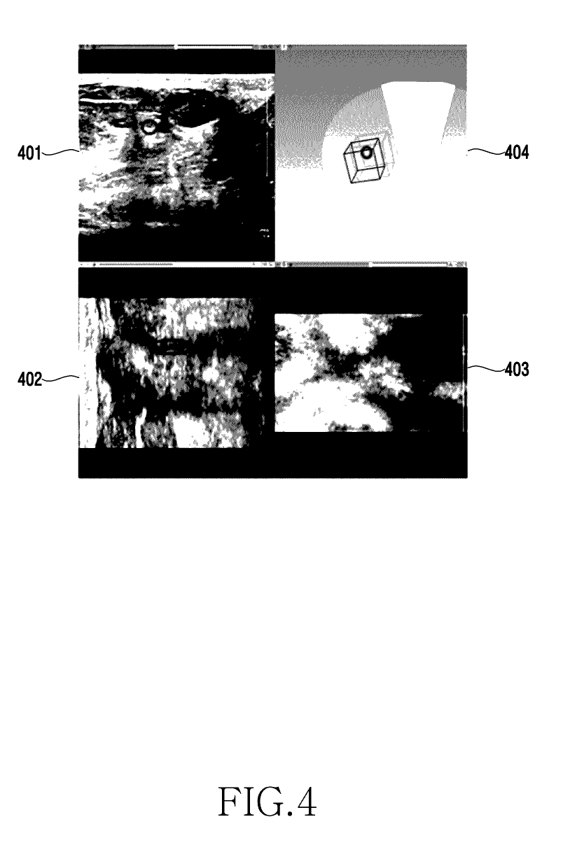

FIG. 4 illustrates an example of displaying position information of an ultrasound image on an ultrasonographic image and a 3D model. The ultrasonographic image is accurate but is used to perform an examination on only a part of the human body rather than image-capturing the whole of the human body, position information, and thus requires position information on a part of the human body in which the ultrasonographic image is captured. Referring to FIG. 4, there are three ultrasonographic cross-sectional images captured in directions of length 401, width 402, and verticality 403. Also, in the three ultrasonographic cross-sectional images, an area-of-interest exists in a part represented in the form of a circle. In FIG. 4, the two ultrasonographic cross-sectional images 401 and 402 include one acquired identical area-of-interest. Corresponding coordinates of the second image on a 3D model may be calculated in the right upper view 404, and an image-capturing range of the second image having a rectangular shape may be displayed on the 3D model. Coordinates of the collected area-of-interest on the 3D model are displayed on the 3D model, and in FIG. 4, the coordinates of the collected area-of-interest are represented in the form of a circle. Accordingly, it can be confirmed from the right upper view 404 that the area-of-interest has been acquired in the second image.

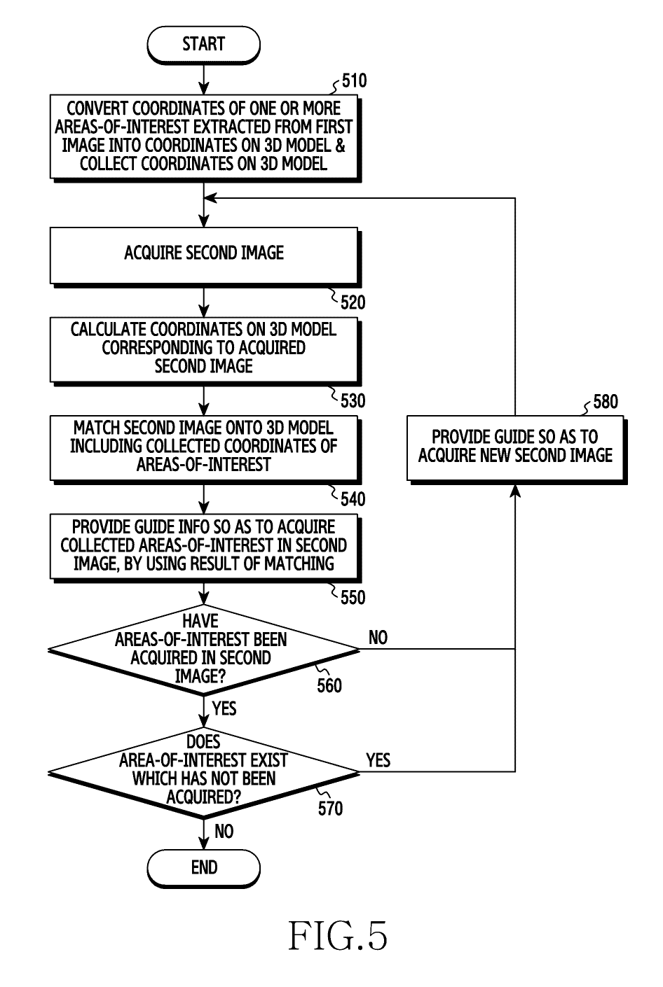

FIG. 5 is a flowchart illustrating a method for supporting acquisition of an area-of-interest in an ultrasound image according to an embodiment of the present disclosure. Referring to FIG. 5, a description will be made of the method for supporting acquisition of an area-of-interest in an ultrasound image by the apparatus 100 for supporting acquisition of an area-of-interest in an ultrasound image according to an embodiment of FIG. 1.

First, coordinates of one or more areas-of-interest extracted from a first image may be converted into coordinates on a 3D model and the coordinates on the 3D model of one or more areas-of-interest may be collected in operation 510, and the 3D model may be output on the screen in operation 510. At this time, when the first image is a 3D image, a coordinate system of a measurement device that has captured the first image may be converted into a coordinate system of the 3D model, and thereby, the coordinates of the areas-of-interest may be converted into coordinates on the 3D model.

When the first image is a 2D image, 2D coordinates of an area-of-interest included in a cross-sectional image may be converted into coordinates on the 3D model. A predetermined area may be determined as an area-of-interest, with the converted coordinates on the 3D model as a center. At this time, the predetermined area may be a straight line on the 3D model formed perpendicular to a cross section corresponding to the coordinates of the area-of-interest, with the coordinates of the area-of-interest as a center.

When there are two or more 2D cross-sectional images obtained by image-capturing an identical area-of-interest, coordinates of an intersection point of straight lines on the 3D model, which are respectively formed perpendicular to the cross-sectional images, may be collected as coordinates of the area-of-interest. When an intersection point is not formed on the basis of 2D coordinates of the area-of-interest, a predetermined area may be determined as an area-of-interest with coordinates of the area-of-interest on the 3D model, into which the coordinates of the area-of-interest included in each 2D cross-sectional image are converted, as a center. At this time, the predetermined area may include one or more of areas within a preset radius with the coordinates of the area-of-interest, which are converted into the coordinates on the 3D model, as a center. Various embodiments of the predetermined area may be implemented, and thus, embodiments of the predetermined area are not limited thereto.

Then, the 3D model may be output on the screen, and the areas-of-interest may be displayed on the 3D model by using one or more of a point, a line, a plane, and a polygon on the basis of the converted coordinates on the 3D model of the one or more areas-of-interest. At this time, with respect to the collected areas-of-interest, area-of-interest information is collected which includes one or more of the number of the collected areas-of-interest, grades thereof, types thereof, and attributes thereof, and the collected area-of-interest information may be further displayed at a predetermined position on the 3D model.

Next, a second image is acquired by an ultrasound image measurement device in operation 520. When the second image has been acquired, coordinates on the 3D model corresponding to the acquired second image are calculated in operation 530. Then, the second image may be matched onto the 3D model including the collected coordinates of the areas-of-interest in operation 540.

Then, by using a result of the matching, guide information may be provided to the user so as to acquire the collected areas-of-interest in the second image, in operation 550. At this time, as an example, in operation 560, the areas-of-interest are marked and displayed on the 3D model, and a determination is made as to whether the areas-of-interest have been acquired in the second image, on the basis of the converted coordinates of the areas-of-interest and the calculated coordinates of the second image. When it is determined that the areas-of-interest have not been acquired in the second image, the user may be guided to acquire a new second image, in operation 580.

When the areas-of-interest have been acquired in the second image, a determination is made as to whether an area-of-interest exists which has not been acquired, in operation 570. When the area-of-interest exists which has not been acquired in the second image, the user may be guided to acquire a new second image, in operation 580. Since the position of the second image may be changed, the user may be guided to change the position of the second image and acquire the area-of-interest. More specifically, the guide information provided to the user may include one or more pieces of information among an order of acquisition of areas-of-interest, position information of the second image, the degree of proximity of the second image to an area-of-interest, a progress direction of the second image, the number of areas-of-interest which have not been acquired, and position information of the areas-of-interest which have not been acquired.

Also, according to an embodiment of the present disclosure, when the number of areas-of-interest is plural, an order of areas-of-interest required to be acquired may be calculated according to grades of the areas-of-interest, importances thereof, attributes thereof, types thereof, and the like, and may be provided to the user. The areas-of-interest may be provided to the user so as to be marked and include coordinates or additional information, or may have the degree of proximity of the second image to the areas-of-interest according to the proximity of the second image to the areas-of-interest, a current progress direction of the second image, and a guided progress direction of the second image, which are displayed in the areas-of-interest. Also, a determination may be made as to whether an area-of-interest has been acquired in the second image; the user may be provided with the number of areas-of-interest which have not been acquired, coordinate information on an area-of-interest required to be acquired next, and the like; and position information of the areas-of-interest which have not been acquired may be guided to the user.

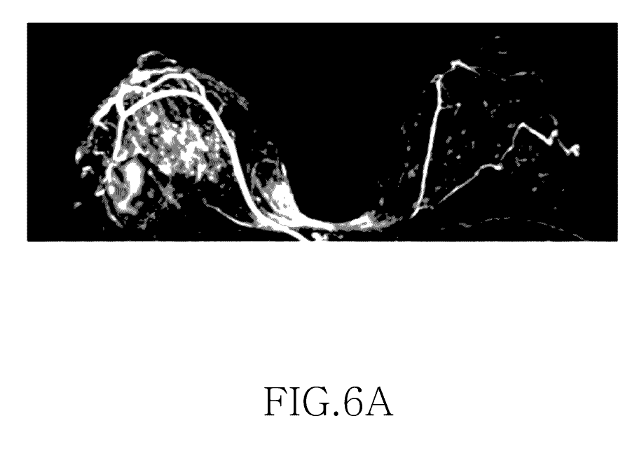

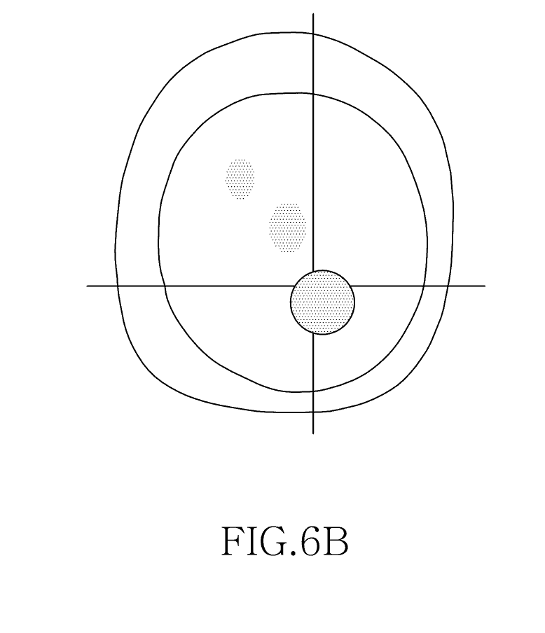

FIG. 6A illustrates an example of displaying an area-of-interest on a translucent 3D model.

The apparatus 100 for supporting acquisition of an area-of-interest in an ultrasound image may use a visually-distinguishable indication so as to be capable of acquiring an area-of-interest in a second image. The 3D model having an area-of-interest displayed thereon may be used in a breast examination. At this time, referring to FIG. 6A, the 3D model which coincides with a body proportion may be displayed in a translucent form, and the area-of-interest may be displayed to be clearly distinguished from others in a visual manner by using a different color of the area-of-interest. Alternatively, the area-of-interest may be displayed on the 3D model by changing a color, or a human body may be displayed by using an outline, and an area to be examined and the area-of-interest may be displayed in different colors. Alternatively, the area to be examined and the area-of-interest may be displayed by using contours having different colors according to the depth of the 3D model. Various embodiments thereof may be implemented.

FIG. 6B illustrates an example in which the apparatus 100 for supporting acquisition of an area-of-interest in an ultrasound image provides guide information on a progress direction of a second image onto a 3D model having an area-of-interest displayed thereon. The ultrasound image measurement device performs ultrasonography in a state where a probe is contacting the surface of the human body. Referring to FIG. 6B, the area-of-interest is displayed on the 3D model representing breasts of a woman. When the apparatus 100 for supporting acquisition of an area-of-interest in an ultrasound image translucently displays the surface of the human body as the 3D model, the area-of-interest located inside the 3D model is projected, and thus, the user may be provided with guide information on the surface of the human body at which the area-of-interest is located. At this time, an area represented by an intersection point of horizontal and vertical straight lines may be considered as a point which needs to be examined in order to acquire an area-of-interest, and the probe may be guided to be capable of measuring this part. At this time, coordinates at which the probe needs to make contact may be guided, a progress direction of the probe may be guided, or coordinates of the acquired area-of-interest and those of an area-of-interest required to be acquired may be provided to the user. However, this configuration is for illustrative purposes only, and thus, the present disclosure is not limited thereto.

The apparatuses, components, and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

The processes, functions, and methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, Wi-Fi, etc.). In addition, functional programs, codes, and code segments for accomplishing the example disclosed herein can be construed by programmers skilled in the art based on the flow diagrams and block diagrams of the figures and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

Meanwhile, the present embodiments can be implemented in the form of computer-readable codes in a computer-readable recording medium. The computer-readable recording medium includes all types of recording devices in which data readable by a computer system are stored.

Examples of the computer-readable recording medium include a ROM, a RAM, a CD-ROM, a magnetic tape, a floppy disk, an optical data storage device, and the like, and include a medium implemented in the form of a carrier wave (e.g., transmission through the Internet). In addition, computer-readable recording media may be distributed over computer systems connected by a network, so that computer-readable codes can be stored and executed in a distributed manner. Further, functional programs, codes and code segments for the implementation of the embodiments may be easily inferred by programmers in the art which the present disclosure pertains to.

1. An apparatus for supporting acquisition of an area-of-interest in an ultrasound image, the apparatus comprising:
at least one processor configured to:
convert coordinates of one or more areas-of-interest extracted from a first image into coordinates on a three-dimensional (3D) model, and to collect coordinates on the 3D model of the one or more areas-of-interest;
calculate coordinates on the 3D model corresponding to an acquired second image when the second image is acquired, and to match the second image onto the 3D model including the collected coordinates of the areas-of-interest; and
provide guide information so as to acquire the collected areas-of-interest in the second image, by using a result of the matching.

2. The apparatus of claim 1, wherein the first image corresponds to a radiographic image, and the second image corresponds to an ultrasonographic image.

3. The apparatus of claim 1, wherein, when the first image corresponds to a 3D image, the at least one processor converts a coordinate system of a measurement device, that has captured the first image, into a coordinate system of the 3D model, and thereby converts the coordinates of the one or more areas-of-interest into the coordinates on the 3D model.

4. The apparatus of claim 1, wherein, when the first image corresponds to two or more 2D cross-sectional images obtained by image-capturing an identical area-of-interest, the at least one processor collects, as the coordinates of the area-of-interest, coordinates of an intersection point of straight lines on the 3D model which are respectively formed perpendicular to the cross-sectional images corresponding to the 2D coordinates of the area-of-interest, which is included in at least some of the two or more 2D cross-sectional images, with the 2D coordinates of the area-of-interest as a center.

5. The apparatus of claim 1, wherein, when the first image corresponds to a 2D image and an intersection point is not formed based on coordinates of an area-of-interest included in at least some 2D cross-sectional images among the one or more first images, the at least one processor determines, as an area-of-interest, a predetermined area which is converted into coordinates on the 3D model with the coordinates of the area-of-interest, which is included in the at least some 2D cross-sectional images, as a center.

6. The apparatus of claim 5, wherein the predetermined area comprises one or more of:
straight lines which are converted into coordinates on the 3D model and which are respectively formed perpendicular to cross-sectional images corresponding to the coordinates of the area-of-interest, which is included in the at least some 2D cross-sectional images, with the coordinates of the area-of-interest as the center; and
an area within a preset radius with the converted coordinates of the areas-of-interest as a center.

7. The apparatus of claim 1, further a display configured to output the 3D model on a screen, and to display the one or more areas-of-interest on the output 3D model in a preset form including one or more of a point, a line, a plane, and a polygon based on the collected coordinates of the areas-of-interest.

8. The apparatus of claim 7, wherein display displays the 3D model in at least one form among preset forms including one or more of translucency and a contour.

9. The apparatus of claim 7, wherein the at least one processor collects area-of-interest information including one or more of the number of the one or more areas-of-interest, grades thereof, types thereof, and attributes thereof, and the display further displays the collected area-of-interest information at a predetermined position on the 3D model.

10. The apparatus of claim 1, wherein the at least one processor determines whether an area-of-interest exists which has not been acquired in the second image among the one or more areas-of-interest, based on the converted coordinates of the areas-of-interest and the calculated coordinates of the second image, and provides the guide information to a user so as to acquire a new second image, when it is determined that the area-of-interest exists which has not been acquired in the second image.

11. The apparatus of claim 1, wherein the at least one processor displays an arrow indicating an area-of-interest which has not been acquired in the second image, or emphasizes and displays the area-of-interest, which has not been acquired in the second image, by using one or more of a type of an edge color of an outline, a line type of the outline, and a line thickness of the outline.

12. The apparatus of claim 1, wherein the guide information comprises one or more pieces of information among an order of the acquisition of the areas-of-interest, position information of the second image, a degree of proximity of the second image to the areas-of-interest, a progress direction of the second image, the number of areas-of-interest which have not been acquired, and position information of the areas-of-interest which have not been acquired.

13. A method for supporting acquisition of an area-of-interest in an ultrasound image, the method comprising:
converting coordinates of one or more areas-of-interest extracted from a first image into coordinates on a three-dimensional (3D) model, and collecting coordinates on the 3D model of the one or more areas-of-interest;
calculating coordinates on the 3D model corresponding to an acquired second image when the second image is acquired;
matching the second image onto the 3D model including the collected coordinates of the areas-of-interest; and
providing guide information to a user so as to acquire the one or more areas-of-interest in the second image, by using a result of the matching.

14. The method of claim 13, wherein the collecting of the coordinates on the 3D model of the one or more areas-of-interest comprises, when the first image corresponds to a 3D image, converting a coordinate system of a measurement device, that has captured the first image, into a coordinate system of the 3D model, and thereby converting the coordinates of the one or more areas-of-interest into the coordinates on the 3D model.

15. The method of claim 13, wherein the collecting of the coordinates on the 3D model of the one or more areas-of-interest comprises, when the first image corresponds to two or more 2D cross-sectional images obtained by image-capturing an identical area-of-interest, collecting, as the coordinates of the area-of-interest, coordinates of an intersection point of straight lines on the 3D model which are respectively formed perpendicular to cross-sectional images corresponding to the 2D coordinates of the area-of-interest, which is included in at least some of the two or more 2D cross-sectional images, with the 2D coordinates of the area-of-interest as a center.

16. The method of claim 13, wherein the collecting of the coordinates on the 3D model of the one or more areas-of-interest comprises, when the first image corresponds to a 2D image and an intersection point is not formed based on coordinates of an area-of-interest included in at least some 2D cross-sectional images among the one or more first images, determining, as an area-of-interest, a predetermined area which is converted into coordinates on the 3D model with the coordinates of the area-of-interest, which is included in the at least some 2D cross-sectional images, as a center.

17. The method of claim 16, wherein the predetermined area comprises one or more of:
straight lines which are converted into coordinates on the 3D model and which are respectively formed perpendicular to cross-sectional images corresponding to the coordinates of the area-of-interest, which is included in the at least some 2D cross-sectional images, with the coordinates of the area-of-interest as the center; and
an area within a preset radius with the converted coordinates of the areas-of-interest as a center.

18. The method of claim 13, wherein the collecting of the coordinates on the 3D model of the one or more areas-of-interest comprises:
outputting the 3D model on a screen; and
displaying the one or more areas-of-interest on the output 3D model in a preset form including one or more of a point, a line, a plane, and a polygon based on the collected coordinates of the areas-of-interest;
collecting area-of-interest information including one or more of the number of the one or more areas-of-interest, grades thereof, types thereof, and attributes thereof; and
further displaying the collected area-of-interest information at a predetermined position on the 3D model.

19. (canceled)

20. The method of claim 13, wherein the providing of the guide information comprises:
determining whether an area-of-interest exists which has not been acquired in the second image among the one or more areas-of-interest, based on the converted coordinates of the areas-of-interest and the calculated coordinates of the second image; and
providing a guide so as to acquire a new second image, when it is determined that the area-of-interest exists which has not been acquired in the second image.

21. The method of claim 13, wherein the providing of the guide information comprises displaying an arrow indicating an area-of-interest which has not been acquired in the second image, or emphasizing and displaying one or more of a type of an edge color of an outline of the area-of-interest which has not been acquired in the second image, a line type of the outline of the area-of-interest, and a line thickness of the outline of the area-of-interest.
